(19) United States
(12) Patent Application Publication
(10) Pub. No.: US 2025/0090534 A1
(43) Pub. Date: Mar. 20, 2025

(54) Phosphodiesterase-5 Inhibitor Combinations, Methods of Making, and Methods of Use Thereof

(71) Applicant: VK Research Associates Inc., Dover, DE (US)

(72) Inventors: Siva Rama Krishna NUTALAPATI, Princeton, NJ (US); Victor CHEHEBAR, Massapequa, NY (US)

(21) Appl. No.: 18/948,795

(22) Filed: Nov. 15, 2024

Related U.S. Application Data
(63) Continuation of application No. 17/190,774, filed on Mar. 3, 2021, now abandoned.
(60) Provisional application No. 62/985,096, filed on Mar. 4, 2020.

(51) Int. Cl. A61K 31/519; A61K 9/00; A61K 9/20; A61K 9/24; A61K 9/48; A61K 31/135; A61K 31/138; A61K 31/167; A61K 31/192; A61K 31/195; A61K 31/407; A61K 31/415; A61K 31/4439; A61K 31/4985; A61K 31/616; A61K 31/80; A61K 33/00; A61K 33/10; A61K 47/02; A61K 47/12; A61K 47/26; A61K 47/36

(52) U.S. Cl. CPC A61K 31/519; A61K 9/0053; A61K 9/0095; A61K 9/2009; A61K 9/2013; A61K 9/2018; A61K 9/2027; A61K 9/2054; A61K 9/2068; A61K 9/209; A61K 9/2095; A61K 9/4808; A61K 9/4858; A61K 9/4866; A61K 31/135; A61K 31/138; A61K 31/167; A61K 31/192; A61K 31/195; A61K 31/407; A61K 31/415; A61K 31/4439; A61K 31/4985; A61K 31/616; A61K 31/80; A61K 33/00; A61K 33/10; A61K 47/02; A61K 47/12; A61K 47/26; A61K 47/36

(57) ABSTRACT

Combination oral formulations comprising a phosphodiesterase-5 inhibitor and an additional active agent that can reduce or eliminate a side effect associated with phosphodiesterase-5 inhibitor therapy are described. Methods of treatment using the combinations and kit formulations are included.

### CROSS-REFERENCE TO RELATED APPLICATIONS

This patent application is a continuation of U.S. application Ser. No. 17/190,774 filed Mar. 3, 2021 which claims the benefit of priority from U.S. Provisional Application Ser. No. 62/985,096 filed Mar. 4, 2020, each of which is incorporated herein by reference in its entirety for all purposes.

### BACKGROUND

Phosphodiesterase 5 inhibitors (“PDE5 inhibitors”) were initially investigated for the treatment of hypertension and angina. Their use for the treatment of erectile dysfunction (“ED”) grew out of this initial research. The prevalence of erectile dysfunction is very common, where it is estimated that about 320 million men will encounter the disorder by 2025.

Current US FDA-approved PDE5 inhibitors include sildenafil, vardenafil, tadalafil, and avanafil. Other non-FDA, commercially available drugs include lodenafil, udenafil, and mirodenafil.

PDE5 inhibitors are useful in the treatment of a number of conditions, both for chronic use (e.g., dosing daily) or for more limited administration. The various conditions treated or uses include:
1. Erectile Dysfunction (“ED”)
2. Idiopathic Pulmonary Hypertension (“IPAH” or “PAH”)
3. Premature Ejaculation (“PE”) associated with erectile dysfunction
4. High altitude illness, to reduce the incidence of pulmonary hypertension in people with altitude-induced high pulmonary systolic arterial pressure
5. Penile rehabilitation after Radical Prostatectomy
6. Antianginal
7. Lower Urinary Tract Symptoms (“LUTS”) due to Benign Prostatic Hyperplasia (“BPH”).

Additional conditions where PDE5 inhibitor use may be beneficial include the following, several of which require chronic, daily dosing:
1. Heart Failure
2. Stroke
3. Peripheral neuropathy
4. Improving male fertility
5. Peripheral Arterial Disease (“PAD”)
6. Diabetic Nephropathy.

Although all PDE5 inhibitors have similar modes of action, they each exhibit distinct biochemical properties, pharmacokinetic profiles, and clinical performance. The most distinct property is their half-life. Sildenafil, vardenafil, and avanafil have half-lives of about 4 to about 5 hours, compared with tadalafil, with a half-life of 17.5 hours. These PDE5 inhibitors are rapidly absorbed after oral administration with a median time to maximum concentration (t max) of 30 minutes for avanafil (at doses of 50, 100, and 200 mg), 1 hour for sildenafil and vardenafil, and 2 hours for tadalafil. Absorption profiles of sildenafil or vardenafil are affected when administered with a high-fat meal, but are not found with tadalafil or avanafil. Thus, each individual PDE5 inhibitor exhibits different pharmacokinetic properties.

Side effects associated with PDE5 inhibitor therapy have been reported. Two of the most common side effects with use of PDE5 inhibitors are headache and dyspepsia (indigestion). Other side effects associated with PDE5 therapy include back pain, myalgia, migraine, gastritis, and heartburn. These side effects are more common with longer lasting PDE5 inhibitors causing discomfort, annoyance, and even discontinuation of use.

In clinical studies, sildenafil produced side effects of headache and dyspepsia in a dose dependent manner, where these side effects increased with increased dosing:
Headache: 25 mg (16%), 50 mg (21%), 100 mg (28%), Placebo (2%);
Dyspepsia: 25 mg (3%), 50 mg (9%), 100 mg (18%), Placebo (2%).

Side effects associated with sildenafil citrate tend to be underestimated (Moreira Jr. et al. Urology, Vol. 56, Issue 3, 474-476, 2000).

Clinical studies showed tadalafil also produced side effects of headache and dyspepsia in a dose dependent manner:
Headache: 5 mg (11%), 10 mg (11%), 20 mg (15%), Placebo (5%);
Dyspepsia: 5 mg (4%), 10 mg (8%), 20 mg (10%), Placebo (1%).

In an article from Taylor et al. “Differences In Side Effect Duration and Related Bother Levels Between Phosphodiesterase Type 5 Inhibitors” BJUI, 103, 1392-1395, 2008, three PDE5 inhibitors were studied at maximum doses: sildenafil, tadalafil, and vardenafil. For headache, the Durations/Mean Durations for the three PDE5 inhibitors were reported:
Sildenafil 15.6 hours/Mean 4.5 hours
Tadalafil 24.3 hours/Mean 17.0 hours
Vardenafil 18.2 hours/Mean 3.9 hours.

For dyspepsia, the Durations/Mean Durations for the three PDE5 inhibitors were reported:
Sildenafil 10.3 hours/Mean 5.5 hours
Tadalafil 15.5 hours/Mean 11.2 hours
Vardenafil 22.0 hours/Mean 6.1 hours.

Visual Analogue Scale (VAS) Scores For Individual Side Effect Bother (100% scale) Headache 35.7% and Dyspepsia 46.2%.

Variation in the Duration of Symptoms or Bother Scale between the three inhibitors were due to their differing pharmacokinetic profiles.

Data was accumulated from the patients at 3 months follow up visits: Employed for the Bother Scale: VAS scale from 0-100%.

The study concluded that headache and dyspepsia were more common in tadalafil versus sildenafil, and patients taking tadalafil have more prolonged side effects than those taking sildenafil, which is consistent with their respective half-lives.

Thus, there remains a need in the art for methods and treatments that can lessen the severity of, or even prevent, side effects associated with PDE5 inhibitor therapy. Since the onset, severity and duration of these side effects are PDE5 inhibitor specific, there exists a need to tailor the treatment that is specific to each PDE5 inhibitor and the particular adverse effect caused by it.

### SUMMARY

In an embodiment, a combination oral formulation comprises a phosphodiesterase-5 inhibitor and an additional active agent that can reduce or eliminate a side effect associated with phosphodiesterase-5 inhibitor, wherein the additional active agent is an analgesic, an antacid, an anti-migraine agent, a H2 blocker, a proton pump inhibitor, an anti-gas agent, or a combination thereof.

In another embodiment, a kit comprises the combination oral formulation.

In yet another embodiment, a method of treating a condition treatable with phosphodiesterase-5 inhibitor therapy, comprises administering a combination oral formulation comprising a phosphodiesterase-5 inhibitor and an additional active agent to a patient in need of phosphodiesterase-5 inhibitor therapy thereof; wherein the additional active agent is an analgesic, an antacid, an anti-migraine agent, a H2 blocker, a proton pump inhibitor, an anti-gas agent, or a combination thereof; and wherein the combination results in a reduction or elimination of a side effect associated with phosphodiesterase-5 inhibitor therapy alone.

These and other features and characteristics are more particularly described below.

### DETAILED DESCRIPTION

Disclosed herein are combination oral therapies that allow for the convenience of receiving PDE5 inhibitor therapy while at the same time eliminating, or significantly reducing the severity of, side effects commonly associated with PDE5 inhibitor therapy, including the most bothersome side effects headache and dyspepsia, as well as back pain, myalgia, gastritis, and heartburn. The combination formulations, kits, and dosing regimens disclosed herein include a PDE5 inhibitor and an additional active agent that provides the necessary relief from adverse symptoms in the convenience of a single oral dosage form comprising all of the active agents, or separate oral dosage forms of each active agent conveniently packaged together for concomitant administration. The combinations have the additional beneficial effect of improving patient compliance with PDE5 inhibitor therapy by reducing or eliminating side effects.

The combination oral therapies disclosed herein allow for a tailoring of the PDE5 inhibitor treatment and control of side effects, taking into account the particular PDE5 inhibitor, its particular pharmacokinetic properties, whether the dosing is chronic or acute, the dosage strength used, and the particular adverse effect caused by the same, including the onset and duration of a particular adverse effect.

Two of the most bothersome side effects from PDE5 inhibitor therapy noted in multiple studies are headache and dyspepsia. Most patients do not carry or have analgesic medications or “antacids” readily available and have experienced one or both of these side effects, which either lead to the discontinuation of these PDE5 inhibitors or resultant pain and suffering.

With sildenafil and tadalafil, for example, side effects of headache and dyspepsia occurred in a dose dependent manner, with higher doses associated with higher incidences of these side effects. Therefore, even if sildenafil and tadalafil are administered acutely to treat ED, these side effects will be prominent especially at higher dosage strengths. Additionally, even if PDE5 inhibitors are administered at a lower dose, when administered chronically, for example for BPH or PAH, daily administration will still result in the patient experiencing side effects.

In an embodiment, the combination regimens disclosed herein would be a very important advancement in the treatment of ED when using PDE5 inhibitors to achieve erection so as to alleviate pain and suffering associated with the prevalent side effects, especially headache and dyspepsia, but also back pain, myalgia, gastritis, and heartburn. In addition, the combinations disclosed herein would be an important advancement in the treatment of benign prostatic hyperplasia (BPH), idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, to alleviate the side effects associated with PDE5 inhibitor use particularly in view of the chronic, daily use of such inhibitors in many of these conditions. Symptomatic use includes, for example, treatment of ED, high altitude illness, and the like; chronic used includes, for example, treatment of BPH, idiopathic pulmonary hypertension, premature ejaculation associated with erectile dysfunction, penile rehabilitation after radical prostatectomy, angina pectoris, heart failure, stroke, peripheral neuropathy, male infertility, peripheral arterial disease, diabetic nephropathy, and the like.

The combinations comprise a PDE5 inhibitor and an additional active agent that can reduce or eliminate a side effect associated with PDE5 inhibitor therapy, whether from acute dosing or chronic dosing. In one embodiment, the combinations can reduce or eliminate a side effect associated with acute PDE5 inhibitor therapy, for example the treatment of ED, and specifically treatment of ED with high doses of PDE5 inhibitors. In another embodiment, the combinations can reduce or eliminate a side effect associated with chronic PDE5 inhibitor therapy, for example in the treatment of BPH or idiopathic pulmonary hypertension, and specifically treatment of with daily doses of PDE5 inhibitors. The combination therapy disclosed herein would enhance the quality of life of the patient in need of PDE5 inhibitor therapy by significantly diminishing or eliminating these side effects.

In the treatment of ED, some patients require higher doses of PDE5 inhibitors for efficacy than other patients. For acute dosing, since side effects such as headaches and gastritis were found to be dose related for PDE5 inhibitors sildenafil and tadalafil, the combinations herein make available ED treatment to this subpopulation of patients that would otherwise forego such treatment in order to avoid the side effects at the elevated doses.

The side effects can be significantly reduced or eliminated in a preventative manner, that is, the additional active agent can be provided before or immediately prior to onset of a side effect associated with PDE5 inhibitor therapy in the absence of the additional active agent. The release of the additional active agent can be controlled so that the additional active agent is released at the appropriate time or for an appropriate period of time when a side effect would be expected to occur when the PDE5 inhibitor is administered in the absence of the additional active agent. For example, oral tadalafil therapy results in both side effects having an early onset and long duration, e.g. headache, dyspepsia, and myalgia, as well as side effects having delayed onset and a short duration (up to 3 hours), e.g., gastritis and back pain. The additional active agent in a combination of tadalafil as the PDE5 inhibitor, for example, can be selected to target a specific side effect and formulated to effectively treat or prevent that side effect in terms of both onset and duration.

The additional active agent to be used in combination with a PDE5 inhibitor can be an analgesic, an antacid, an anti-migraine agent, a H2 blocker, a proton pump inhibitor, an anti-gas agent, or a combination thereof. Each of the PDE5 inhibitor and the additional active agent (i.e., analgesic, antacid, H2 blocker, proton pump inhibitor, anti-gas agent, or a combination thereof) can be administered as an immediate release dosage form, as a controlled release (e.g., extended release) dosage form, or a combination of immediate release and controlled release dosage form.

Appropriate combinations can be achieved by correlating the therapeutic effect of the additional active agent with the half-life of the particular PDE5 inhibitor used, specifically by tailoring the release and duration of the additional active agent through tailored formulating. For example, oral administration of PDE5 inhibitor avanafil has a half-life of about 4 to 5 hours and achieves a median time to maximum concentration at about 30 minutes. Any side effect from acute avanafil therapy would appear quickly and last for a few hours. Thus, it would be advantageous to have the additional active agent, such as an analgesic, in an immediate release form in order to effectively manage a side effect from avanafil treatment including headache, migraine, or myalgia. In another example, oral administration of tadalafil has a half-life of 17.5 hours and achieves a median time to maximum concentration at about 2 hours. Any side effect from acute tadalafil therapy would appear later compared to avanafil and continue for several hours longer. When tadalafil is the PDE5 inhibitor it may be more appropriate to have the analgesic additional active agent in an extended release form in order to effectively manage a reduction or elimination of a side effect from tadalafil treatment, which would be expected to last for a longer duration.

Therefore, the release of the additional active agent from the combination can be tailored depending upon the PDE5 inhibitor type and strength used in the combination and the particular adverse effect to be targeted. Use of an extended release analgesic, such as extended release acetaminophen or naproxen, can be used to prevent or lessen headache, back pain, and myalgia side effects caused by tadalafil and sildenafil. Mild headaches can be addressed with shorter acting combinations of analgesics and PDE5 inhibitors such as an immediate release form of each active agent.

For a combination of immediate release and controlled release dosage form, the PDE5 inhibitor can be immediate release and each additional active agent can be controlled release; the PDE5 inhibitor can be controlled release and each additional active agent individually can be immediate release, controlled release, or a combination thereof; a first portion of the PDE5 inhibitor can be administered as immediate release and a second portion can be administered as controlled release, and each additional active agent individually can be immediate release, controlled release, or a combination thereof; or the PDE5 inhibitor can be immediate release, controlled release, or a combination thereof and each additional active agent individually can be administered with a first portion as immediate release and a second portion as controlled release.

In an embodiment, the combination of a PDE5 inhibitor and an additional active agent that is an analgesic, an anti-migraine agent, or a combination thereof can be used to reduce or eliminate the side effect of headache, migraine, back pain, myalgia, or a combination thereof.

Headaches that result from use of PDE5 inhibitors are vascular in nature and therefore, migraine sufferers will be more prone to these headaches. For frequent migraine suffers that may already be taking prophylactic medications to prevent their headaches, such as a beta blocker, they may benefit from a combination of a PDE5 inhibitor and an analgesic to minimize the onset or severity of these migraines, while reducing the need for higher doses of the prophylactic beta blocker and their accompanying side effects.

In an embodiment, the combination of a PDE5 inhibitor and an additional active agent that is an antacid, a H2 blocker, a proton pump inhibitor, or a combination thereof can be used to reduce or eliminate the side effect of dyspepsia, gastritis, heartburn, or a combination thereof. Within this embodiment, an anti-gas agent can be included in the combination.

PDE5 inhibitors that can be used in the combination include, for example, avanafil, sildenafil, tadalafil, vardenafil, lodenafil, udenafil, mirodenafil, and the like, or a pharmaceutically acceptable salt thereof or a combination thereof. Exemplary PDE5 inhibitor salts include sildenafil citrate, vardenafil monohydrochloride, lodenafil carbonate, and the like, or a combination thereof.

Avanafil or a pharmaceutically acceptable salt thereof can be used in the combination in an amount of about 10 milligrams (mg) to about 300 mg base equivalent per dosage form, specifically about 50 mg, about 100 mg, about 150 mg, about 200 mg, or about 250 mg base equivalent. Sildenafil or a pharmaceutically acceptable salt thereof can be used in the combination in an amount of about 10 mg to about 200 mg base equivalent per dosage form, specifically about 20 mg, about 25 mg, about 50 mg, about 75 mg, about 100 mg, or about 150 mg base equivalent. Tadalafil or a pharmaceutically acceptable salt thereof can be used in the combination in an amount of about 1.0 mg to about 50 mg base equivalent per dosage form, specifically about 2.0 mg, about 2.5 mg, about 5.0 mg, about 10 mg, about 15 mg, about 20 mg, about 30 mg or about 40 mg base equivalent. Vardenafil or a pharmaceutically acceptable salt thereof can be used in the combination in an amount of about 1.0 mg to about 50 mg base equivalent per dosage form or as the monohydrochloride, specifically about 2.0 mg, about 2.5 mg, about 4.0 mg, about 5.0 mg, about 8.0 mg about 10 mg, about 15 mg, about 20 mg, about 30 mg or about 40 mg base equivalent or as the monohydrochloride. Lodenafil or a pharmaceutically acceptable salt thereof can be used in the combination in an amount of about 10 mg to about 200 mg base equivalent per dosage form, specifically about 40 mg, about 80 mg, about 100 mg, or about 160 mg base equivalent or as the carbonate. Udenafil or a pharmaceutically acceptable salt thereof can be used in the combination in an amount of about 10 mg to about 200 mg base equivalent per dosage form, specifically about 40 mg, about 80 mg, about 100 mg, or about 160 mg base equivalent. Mirodenafil or a pharmaceutically acceptable salt thereof can be used in the combination in an amount of about 10 mg to about 200 mg base equivalent per dosage form, specifically about 50 mg, about 75 mg, about 100 mg, or about 150 mg base equivalent.

For the treatment of headaches, migraine, back pain, or myalgia, the combination can comprise a PDE5 inhibitor and an analgesic such as non-steroidal, anti-inflammatory (NSAID) drug. In one embodiment, the combination comprises a PDE5 inhibitor and an analgesic, specifically a NSAID, each of which are formulated in an immediate release dosage form, an extended release dosage form, or a combination thereof. In one embodiment, the combination comprises a PDE5 inhibitor formulated for immediate release form and an analgesic, specifically a NSAID, formulated for extended release.

Suitable analgesies include acemetacin, acetaminophen, aminoprofen, aspirin, benoxaprofen, bucloxic acid, carisoprodol, carprofen, celecoxib, clidanac, cyclobenzaprine, diclofenac, diflurisal, fentiazac, flufenamic acid, flurbiprofen, fenoprofen, flubufen, flufenisal, isoxicam, ketoprofen, ibuprofen, indomethacin, indoprofen, ketorolac, naproxen, nabumetone, meclofenamic acid, meclofenate, mefenamic acid, metaxalone, methocarbamol, muroprofen, niflumic acid, orphenadrine, oxaprozin, oxpinac, piroprofen, piroxicam, pramoprofen, sudoxicam, sulindac, suprofen, tiaprofenic acid, tiopinac, tolmetin, tolfenamic acid, trioxaprofen, zidometacin, zomepirac, and the like, or a pharmaceutically acceptable salt thereof (e.g. naproxen sodium, orphenadrine citrate), or a combination thereof. In an embodiment, the analgesics are in extended release form. In one embodiment, the combination formulation comprises extended release acetaminophen or extended release naproxen sodium.

Acetaminophen can be used in the combination in an amount of about 10 mg to about 1300 mg per dosage form, specifically about 25 mg, about 50 mg, about 80 mg, about 100 mg, about 120 mg, about 160 mg, about 200 mg, about 240 mg, about 300 mg, about 325 mg, about 400 mg, about 500 mg, about 600 mg, about 650 mg, about 700 mg, about 800 mg, about 900 mg, about 1000 mg, about 1100 mg, about 1200 mg, or about 1300 mg per dosage form.

Ibuprofen can be used in the combination in an amount of about 10 mg to about 1300 mg per dosage form, specifically about 25 mg, about 50 mg, about 80 mg, about 100 mg, about 120 mg, about 160 mg, about 200 mg, about 240 mg, about 300 mg, about 325 mg, about 400 mg, about 500 mg, about 600 mg, about 650 mg, about 700 mg, about 800 mg, about 900 mg, about 1000 mg, about 1100 mg, about 1200 mg, or about 1300 mg per dosage form.

Naproxen or a pharmaceutically acceptable salt thereof can be used in the combination in an amount of about 25 mg to about 1000 mg base equivalent per dosage form, specifically about 50 mg, about 60 mg, about 120 mg, about 150 mg, about 200 mg, about 220 mg, about 250 mg, about 300 mg, about 375 mg, about 400 mg, about 500 mg, about 750 mg or about 1000 mg base equivalent per dosage form.

Celecoxib can be used in the combination in an amount of about 25 mg to about 600 mg per dosage form, specifically about 50 mg, about 75 mg, about 100 mg, about 150 mg, about 200 mg, about 250 mg, about 300 mg, about 350 mg, about 400 mg, about 450 mg, about 500 mg, or about 550 mg per dosage form.

For the treatment of dyspepsia, gastritis, heartburn, or a combination thereof, the combination formulation can comprise an H2 blocker, a proton pump inhibitor, an antacid, or a combination thereof.

Suitable histamine-2 receptor antagonists (referred to herein as “H2 blockers” or “H2 antagonists”) that can be used in a combination with a PDE5 inhibitor include cimetidine, famotidine, nizatidine, ranitidine, a pharmaceutically acceptable salt thereof, and the like, or a combination thereof. Exemplary pharmaceutically acceptable salts of an H2 blocker include cimetidine hydrochloride, ranitidine hydrochloride, ranitidine bismuth citrate, and the like, or a combination thereof.

Cimetidine or a pharmaceutically acceptable salt thereof can be used in the combination in an amount of about 25 mg to about 900 mg base equivalent per dosage form, specifically about 50 mg, about 75 mg, about 100 mg, about 150 mg, about 200 mg, about 250 mg, about 300 mg, about 350 mg, about 400 mg, about 450 mg, about 500 mg, about 600 mg, about 700 mg, or about 800 mg base equivalent per dosage form.

Ranitidine or a pharmaceutically acceptable salt thereof can be used in the combination in an amount of about 25 mg to about 600 mg base equivalent per dosage form, specifically about 50 mg, about 75 mg, about 100 mg, about 150 mg, about 200 mg, about 250 mg, about 300 mg, about 350 mg, about 400 mg, about 450 mg, about 500 mg, or about 550 mg base equivalent per dosage form.

Suitable proton pump inhibitors include for example esomeprazole, dexlansoprazole, lansoprazole, omeprazole, pantoprazole, rabeprazole, a pharmaceutically acceptable salt thereof, and the like, or a combination thereof. Exemplary pharmaceutically acceptable salts of a proton pump inhibitor include esomeprazole magnesium, esomeprazole sodium, esomeprazole strontium, omeprazole magnesium, omeprazole sodium, omeprazole strontium, pantoprazole sodium, rabeprazole sodium, and the like, or a combination thereof. In an embodiment, the proton pump inhibitor is formulated for delayed release.

Esomeprazole, omeprazole, pantoprazole, rabeprazole, or a pharmaceutically acceptable salt thereof can be used in the combination in an amount of about 2 mg to about 60 mg base equivalent per dosage form, specifically about 5, about 10 mg, about 20 mg, about 30 mg, about 40 mg, or about 50 mg base equivalent per dosage form.

Suitable antacids that can be used in combination with PDE5 inhibitors include for example a pharmaceutically acceptable alkali or alkaline earth metal carbonate, a pharmaceutically acceptable alkali or alkaline earth metal hydroxide, or a combination thereof. Exemplary antacid carbonates include calcium carbonate, magnesium carbonate, sodium bicarbonate, and the like, or a combination thereof. Exemplary antacid hydroxides include aluminum hydroxide, magnesium hydroxide and the like, or a combination thereof.

Magnesium hydroxide, calcium carbonate, or magnesium carbonate can be used in the combination in an amount of about 50 mg to about 800 mg per dosage form, specifically about 75 mg, about 100 mg, about 150 mg, about 165 mg, about 200 mg, about 300 mg, about 343 mg, about 400 mg, about 500 mg, about 600 mg, or about 700 mg per dosage form.

Aluminum hydroxide can be used in the combination in an amount of about 25 mg to about 200 mg per dosage form, specifically about 50 mg, about 80 mg, about 100 mg, about 125 mg, about 150 mg, or about 160 mg per dosage form.

The PDE5 inhibitor and antacid combination may optionally further include an anti-gas agent such as simethicone.

Simethicone can be used in the combination in an amount of about 25 mg to about 200 mg per dosage form, specifically about 50, about 75 mg, about 100 mg, about 125 mg, about 150 mg, or about 175 mg per dosage form.

PDE5 inhibitors can be used in combination with anti-migraine agents to reduce or eliminate the side effect of headaches caused by PDE5 treatment, for the treatment of migraines, and the like. Suitable anti-migraine agents that can be used in combination with PDE5 inhibitors include for example beta blockers (acebutolol, atenolol, betaxolol, bisoprolol, metoprolol, nadolol, propranolol, timolol, a pharmaceutically acceptable salt thereof), calcium channel blockers (nimodipine, verapamil), antidepressants (amitriptyline, nortriptyline), antiseizure medications (gabapentin, topiramate, valproic acid), calcitonin gene-related peptide “CGRP” inhibitors (erenumab, fremanezumab), and the like, or a combination thereof. In an embodiment, the beta blocker is formulated for extended release.

Exemplary pharmaceutically acceptable salts of an anti-migraine agent include acebutolol hydrochloride, betaxolol hydrochloride, bisoprolol fumarate, metoprolol fumarate, metoprolol succinate, metoprolol tartrate, propranolol hydrochloride, timolol maleate, and the like, or a combination thereof.

Metoprolol or a pharmaceutically acceptable salt thereof can be used in the combination in an amount of about 10 mg to about 350 mg tartrate equivalent per dosage form, specifically about 25 mg, about 37.5 mg, about 50 mg, about 75 mg, about 100 mg, about 150 mg, about 200 mg, about 250 mg, about 300 mg tartrate equivalent per dosage form.

Propranolol or a pharmaceutically acceptable salt thereof can be used in the combination in an amount of about 5 mg to about 200 mg hydrochloride equivalent per dosage form, specifically about 10 mg, about 20 mg, about 25 mg, about 40 mg, about 50 mg, about 60 mg, about 80 mg, about 90 mg, about 100 mg, about 120 mg, or about 160 mg hydrochloride equivalent per dosage form.

An “active agent” means a compound, element, or mixture that when administered to a patient, alone or in combination with another compound, element, or mixture, confers, directly or indirectly, a physiological effect on the patient. When the active agent is a compound, then salts, solvates (including hydrates) of the free compound or salt, crystalline and non-crystalline forms, as well as various polymorphs of the compound are included.

“Pharmaceutically acceptable salt” includes derivatives of an active agent (e.g., PDE5 inhibitor, analgesic, H2 blocker, proton pump inhibitor, antacid, anti-migraine agent, etc.) wherein the active agent is modified by making acid or base addition salts thereof, and further refers to pharmaceutically acceptable solvates, including hydrates, crystalline forms, non-crystalline forms, and polymorphs, of such salts. Examples of pharmaceutically acceptable salts include, but are not limited to, mineral or organic acid addition salts of basic residues such as amines; alkali or organic addition salts of acidic residues; and the like, or a combinations thereof. The pharmaceutically acceptable salts include salts and the quaternary ammonium salts of the active agent. For example, acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, or a combination thereof. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; or a combination thereof.

A “dosage form” means a unit of administration of an active agent, specifically a combination of active agents comprising a PDE5 inhibitor and an additional active agent, for example an analgesic, an anti-migraine agent, an H2 blocker, a proton pump inhibitor, an antacid, and the like, or a combination thereof. Each active agent in the dosage form can have the same release characteristics (e.g., all immediate release), or each active agent can have a separate release characteristic (e.g., immediate release PDE5 inhibitor with an extended release acetaminophen in a bilayer tablet, or immediate release PDE5 inhibitor with a delayed release proton pump inhibitor in a bilayer tablet).

The PDE5 inhibitor combinations can be formulated for oral or buccal administration as a solid, liquid, or semisolid. “Oral dosage form” is meant to include a unit dosage form for oral administration. Exemplary solid oral and buccal dosage forms include tablets, capsules, pellets, films, sachets, powders, chewable gummies, and the like. Exemplary oral liquid dosage forms include solutions, suspensions, emulsions, and the like.

By “immediate-release” is meant a conventional or non-modified release in which greater than or equal to about 75% of the active agent is released within two hours of administration, specifically within one hour of administration, yet more specifically within 30 minutes of administration.

By “controlled-release” is meant a dosage form in which the release of an active agent is controlled or modified over a period of time and does not include immediate-release. Controlled can mean, for example, extended/sustained release, delayed release, or pulsed release at a particular time.

By “sustained release” is meant to include the release of the active agent (e.g., analgesic) at such a rate that blood (e.g., plasma) levels are maintained within a therapeutic range but below toxic levels for about 4 to 24 hours after administration at steady-state. The term “steady-state” means that a plasma level for a given drug has been achieved and which is maintained with subsequent doses of the drug at a level which is at or above the minimum effective therapeutic level and is below the minimum toxic plasma level for a given drug.

By “delayed release” is meant to include the release of the active agent after a predetermined time lag before it begins to release drug.

By “pulsed release” is meant to include an initial quick burst of drug release followed by a predetermined period of no release followed by a second burst of drug release.

The solid, oral dosage form can be a monolithic matrix tablet or a layered tablet having two or more layers wherein the PDE5 inhibitor and the additional active agent can be in separate layers or in the same layer; a capsule; a subunit form such as a plurality of granules, microtablets, minitablets, caplets, pellets (as used herein “pellet” means a spherical granule prepared by extrusion and spheronization, and is equivalent to bead, spheroid, and microsphere), particles, active agent cores, or other multiparticulate system, a plurality of which may optionally be encapsulated, e.g., in a hard gelatin capsule.

The solid, oral dosage form comprises a PDE5 inhibitor, an additional active agent, and a pharmaceutically acceptable excipient. As used herein, “pharmaceutically acceptable excipient” means any other component added to the pharmaceutical formulation other than the active agent. Excipients may be added to facilitate manufacture, enhance stability, enhance product characteristics, enhance bioavailability, enhance patient acceptability, etc. Pharmaceutical excipients include carriers, fillers, binders, disintegrants, lubricants, glidants, granulating agents, compression aids, colors, release controlling agents, sweeteners, preservatives, suspending agents, dispersing agents, film formers, flavorants, printing inks, buffering agents, pH adjusters, preservatives, coatings, and the like. In some instances, a single material will meet two or more of the foregoing general classifications, e.g., depending upon the particular amount of the material employed.

The solid, oral dosage form can comprise about 5 to about 95 weight percent (wt %) total active agent (the PDE5 inhibitor+the additional active agent) based on the total weight of the dosage form, and the balance is a pharmaceutically acceptable excipient; specifically about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt % or about 95 wt % of the total weight of the dosage form or any amount in between.

Exemplary pharmaceutically acceptable excipients include fillers, such as a water insoluble filler, water soluble filler, or a combination thereof. The filler may be a water insoluble filler, such as carnauba wax, stearic acid, silicon dioxide, titanium dioxide, talc, alumina, starch, kaolin, polacrilin potassium, powdered cellulose, microcrystalline cellulose, sodium citrate, dicalcium phosphate, or a combination thereof. Exemplary water-soluble fillers include water soluble sugars and sugar alcohols, specifically lactose, glucose, fructose, sucrose, mannose, dextrose, galactose, the corresponding sugar alcohols and other sugar alcohols, such as mannitol, sorbitol, xylitol, or a combination thereof.

Exemplary binders include alginic acid, a carbomer, carboxymethylcellulose calcium, carboxymethylcellulose sodium, carrageenan, cellulose acetate phthalate, chitosan, ethyl cellulose, guar gum, hydroxyethyl cellulose, hydroxyethylmethyl cellulose, hydroxypropyl cellulose, hydroxypropyl methyl cellulose, methyl cellulose, microcrystalline cellulose, poloxamer, polyethylene oxide, polymethacrylates, polyvinylpyrrolidone (“povidone or PVP”), a saccharide, a starch, partially pregelatinized starch, and the like, or a combination thereof.

Release-retarding material for use in a controlled release solid, oral dosage form matrix formulation can include, for example, a hydrophobic cellulose polymer (e.g. ethylcellulose, hypromellose acetate succinate, cellulose acetate, cellulose acetate propionate, and the like, or a combination thereof); a hydrophilic cellulose polymer (e.g., methylcellulose, hydroxypropylmethylcellulose (HPMC), hydroxypropylcellulose (HPC), hydroxyethylcellulose (HEC), ethylhydroxyethylcellulose (E-HEC), sodium-carboxymethylcellulose (Na-CMC), and the like, or a combination thereof); a non-cellulosic material (e.g., sodium alginate, xanthan gum, carrageenan, chitosan, guar gum, pectin, polyethylene oxide, shellac, zein, and the like, or a combination thereof); a hydrophobic material (e.g., waxes, hydrogenated vegetable oil, hydrogenated castor oil, fatty acids, glyceryl monostearate, stearic acid, and the like); a pharmaceutically acceptable acrylic polymer; or a combination thereof. Generally the hydrophilic polymers and non-cellulosic material used as a controlled release matrix agent are swelling controlled release matrix systems, which control release rate of the active agent by the rate of penetration of media and erosion of the matrix.

Suitable pharmaceutically acceptable acrylic polymer for use as a release-retarding material include, for example, an acrylic acid and methacrylic acid copolymer, a methyl methacrylate copolymer, an ethoxyethyl methacrylate, a cyanoethyl methacrylate, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate, a polymethacrylate, a poly(methyl methacrylate) copolymer, a polyacrylamide, an aminoalkyl methacrylate copolymer, a glycidyl methacrylate copolymer, an ammonio methacrylate copolymer, and the like, or a combination thereof.

Exemplary disintegrants include alginic acid, carboxymethylcellulose calcium, carboxymethylcellulose sodium, cross-linked sodium carboxymethylcellulose (sodium croscarmellose), powdered cellulose, chitosan, croscarmellose sodium, crospovidone, guar gum, low substituted hydroxypropyl cellulose, methyl cellulose, sodium alginate, sodium starch glycolate, partially pregelatinized starch, pregelatinized starch, starch, sodium carboxymethyl starch, and the like, or a combination thereof.

Exemplary lubricants include calcium stearate, magnesium stearate, glyceryl behenate, glyceryl palmitostearate, hydrogenated castor oil, light mineral oil, sodium lauryl sulfate, magnesium lauryl sulfate, sodium stearyl fumarate, stearic acid, zinc stearate, or a combination thereof.

Exemplary glidants include colloidal silica, amorphous silica, precipitated silica, talc, calcium phosphate tribasic, calcium silicate, magnesium silicate, magnesium trisilicate, or a combination thereof.

The solid oral dosage forms can be prepared using equipment and techniques known in the art for tableting included direct compression, granulation, capsule filling, pelletizing, and the like. In common tableting processes, the material which is to be tableted is deposited into a cavity, and one or more punch members are then advanced into the cavity and brought into intimate contact with the material to be pressed, whereupon compressive force is applied. The material is thus forced into conformity with the shape of the punches and the cavity. In a multi-layered embodiment, a bi-layer or tri-layer tablet press may be employed, for example, where each chamber is fed with different a different powder to produce two or three individual layers in the same tablet; or a die is first loaded with the first-layer powder followed by the second-layer powder, that upon compression produces a tablet having two distinct layers (loading a third-layer powder on the second-layer powder can product a tablet with three distinct layers).

The solid, oral dosage form can optionally be coated with a coating for any number of reasons. The coating can be a nonfunctional coating or a functional coating. As used herein, a nonfunctional coating is one that does not substantially affect the overall release profile of the active agent compared to the release profile of the uncoated core formulation. Such coatings can be used for decorative or appearance of the dosage form for identifying purposes (e.g. based on a certain color), patient convenience (case of swallowing), taste-masking, product stability (protection from humidity, light, oxygen), and the like. A functional coating, as used herein, is a coating that provides controlled release of the active agent from the dosage form, including extended/sustained-release, delayed release, or pulsed release. Exemplary non-functional coatings include film coatings. Exemplary functional coatings include enteric coatings and coatings that can target release in a particular region of the gastrointestinal tract. The foregoing coatings may also be used in a multiparticulate system described herein.

In general, a film coating can comprise a water soluble film coating polymer, a plasticizer, a colorant, an additional coating agent such as tale, and the like.

In general, a functional coating can comprise a pH-dependent polymer such as an enteric polymer, a plasticizer, a colorant, an additional coating agent such as tale, and the like. Enteric polymers are predominantly soluble in the intestinal fluid, but substantially insoluble in the gastric juices. Suitable enteric coating polymers include, for example, polyvinyl acetate phthalate (PVAP), hydroxypropylmethyl-cellulose acetate succinate (HPMCAS), cellulose acetate phthalate (CAP), methacrylic acid copolymer, hydroxy propyl methylcellulose succinate, cellulose acetate succinate, cellulose acetate hexahydrophthalate, hydroxypropyl methylcellulose hexahydrophthalate, hydroxypropyl methylcellulose phthalate (HPMCP), cellulose propionate phthalate, cellulose acetate maleate, cellulose acetate trimellitate, cellulose acetate butyrate, cellulose acetate propionate, methacrylic acid/methacrylate polymer (acid number 300 to 330 and also known as EUDRAGIT L, which is an anionic copolymer based on methacrylate and available as a powder, also known as methacrylic acid copolymer, type A (USP NF)), methacrylic acid-methyl methacrylate copolymer, ethyl methacrylate-methylmethacrylate-chlorotrimethylammonium ethyl methacrylate copolymer, methacrylic acid: acrylic acid ethyl ester 1:1 copolymer solid substance of the acrylic dispersion sold under the trade designation “Eudragit L-100-55”, and the like, or a combination thereof. Other functional coating agents include for example, a hydrophobic cellulose polymer (e.g. ethylcellulose, hypromellose acetate succinate, cellulose acetate, cellulose acetate propionate, and the like, or a combination thereof); a hydrophilic cellulose polymer (e.g., methylcellulose, hydroxypropylmethylcellulose (HPMC), hydroxypropylcellulose (HPC), hydroxyethylcellulose (HEC), ethylhydroxyethylcellulose (E-HEC), sodium-carboxymethylcellulose (Na-CMC), and the like, or a combination thereof); a non-cellulosic material (e.g., sodium alginate, xanthan gum, carrageenan, chitosan, guar gum, pectin, polyethylene oxide, shellac, zein, and the like, or a combination thereof); a hydrophobic material (e.g., waxes, hydrogenated vegetable oil, hydrogenated castor oil, fatty acids, glyceryl monostearate, stearic acid, and the like); a pharmaceutically acceptable acrylic polymer; or a combination thereof.

The PDE5 inhibitor combinations can be formulated as a non-chewable, orally disintegrating tablet (alternatively referred to as “orally dispersible tablet”). These dosage forms can be made by methods known to those of ordinary skill in the art of pharmaceutical formulations. For example, Cima Labs has produced oral dosage forms including microparticles and effervescents, which rapidly disintegrate in the mouth and provide adequate taste-masking. Cima Labs has also produced a rapidly dissolving dosage form containing the active agent and a matrix that includes a nondirect compression filler and a lubricant. U.S. Pat. Nos. 5,178,878 and 6,221,392 provide teachings regarding orally disintegrating tablets.

An exemplary orally disintegrating tablet includes a mixture incorporating a water or saliva activated effervescent disintegration agent and the active agent. The mixture may be formulated as a tablet of a size and shape adapted for direct oral administration to a patient. The orally disintegrating tablet is substantially completely disintegrable upon exposure to water or saliva. The effervescent disintegration agent is present in an amount effective to aid in disintegration of the tablet, and to provide a distinct sensation of effervescence when the tablet is placed in the mouth of a patient.

The effervescent sensation is not only pleasant to the patient but also tends to stimulate saliva production, thereby providing additional water to aid in further effervescent action. Thus, once the tablet is placed in the patient's mouth, it will disintegrate rapidly and substantially completely without any voluntary action by the patient. Even if the patient does not chew the tablet, disintegration will proceed rapidly.

The term effervescent disintegration agent includes compounds which evolve gas. Exemplary effervescent disintegration agents evolve gas by means of chemical reactions which take place upon exposure of the effervescent disintegration agent to water or to saliva in the mouth. The bubble or gas generating reaction is most often the result of the reaction of a soluble acid source and an alkali metal carbonate or carbonate source. The reaction of these two general classes of compounds produces carbon dioxide gas upon contact with water included in saliva.

Such water activated materials may be kept in a generally anhydrous state with little or no absorbed moisture or in a stable hydrated form since exposure to water will prematurely disintegrate the tablet. The acid sources or acid may be those which are safe for human consumption and may generally include food acids, acid anhydrides, or acid salts. Food acids include citric acid, tartaric acid, malic acid, fumaric acid, adipic acid, and succinic acids, etc. Because these acids are directly ingested, their overall solubility in water is less important than it would be if the effervescent tablet formulations were intended to be dissolved in a glass of water. An acid anhydride or salt of the above described acids may also be used. Acid salts may include sodium, dihydrogen phosphate, disodium dihydrogen pyrophosphate, acid citrate salts and sodium acid sulfite.

Carbonate sources include dry solid carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate, amorphous calcium carbonate, or a combination thereof.

Where the effervescent agent includes two mutually reactive components, such as an acid source and a carbonate source, in one embodiment both components react substantially completely. Therefore, an equivalent ratio of components which provides for equal equivalents is selected. For example, if the acid used is diprotic, then cither twice the amount of a mono-reactive carbonate base, or an equal amount of a di-reactive base is used for complete neutralization to be realized. However, the amount of either acid or carbonate source may exceed the amount of the other component. This may be useful to enhance taste or performance of a tablet containing an overage of either component. In this case, it is acceptable that the additional amount of either component may remain unreacted.

In general, the amount of effervescent disintegration agent useful for the formation of orally disintegrating tablets is about 5 wt % to about 50 wt % based on the total weight of the final dosage form, specifically about 15 wt % and about 30 wt %, and more specifically about 20 wt % to about 25 wt %.

Other types of orally disintegrating tablets can be prepared without an effervescent agent by using a spray dried carbohydrate or sugar alcohol excipients (e.g. sorbitol, mannitol, xylitol, or a combination thereof, and the like), optionally combined with a disintegrant (e.g. the disintegrant can be crospovidone, croscarmellose, sodium starch glycolate, pregelatinized starch, partially pregelatinized starch, or a combination thereof, and the like), or a glidant (e.g. colloidal silica, silica gel, precipitated silica, or a combination thereof, and the like). Suitable orally disintegrating tablets can be found in U.S. Patent Application Publication US20030118642 A1 to Norman et al. incorporated herein by reference in its entirety.

Orally disintegrating tablets can be manufactured by well-known tableting procedures. In common tableting processes, the material which is to be tableted is deposited into a cavity, and one or more punch members are then advanced into the cavity and brought into intimate contact with the material to be pressed, whereupon compressive force is applied. The material is thus forced into conformity with the shape of the punches and the cavity.

The orally disintegrating tablets typically rapidly disintegrate when orally administered. By “rapid”, it is understood that the tablets disintegrate in the mouth of a patient in less than about 7 minutes, and specifically between about 30 seconds and about 5 minutes, specifically the tablet dissolves in the mouth between about 45 seconds and about 2 minutes. Disintegration time in the mouth can be measured by observing the disintegration time of the tablet in water at about 37° C. The tablet is immersed in the water without forcible agitation. The disintegration time is the time from immersion to substantially complete dispersion of the tablet as determined by visual observation. As used herein, the term “complete disintegration” of the tablet does not require dissolution or disintegration of the subunits or other discrete inclusions. In one embodiment, disintegration can be determined by USP (Test <701>).

In an embodiment, the PDE5 inhibitor combination is formulated as an orally dissolving strip (“film”), which rapidly dissolves in the mouth to release the active agent contained in the strip. The orally dissolving strips generally comprise a water soluble polymer and the active agents. Exemplary classes of water soluble polymers include water soluble cellulosic polymers, water soluble synthetic polymers, water soluble natural gums and polymers or derivatives thereof, or a combination thereof. Exemplary water soluble cellulosic polymers include hydroxypropyl cellulose, hydroxypropylmethyl cellulose, hydroxyethyl cellulose, carboxymethyl cellulose, or a combination thereof. Exemplary water: soluble natural gums and polymers include amylose, dextran, casein, pullulan, gelatin, pectin, agar, carrageenan, xanthan gum, tragacanth, guar gum, acacia gum, arabic gum, sodium alginate, zein, or a combination thereof. Exemplary water soluble synthetic polymers include polyethylene glycol, polyethylene oxide, polyvinyl pyrrolidone, polyvinyl alcohol, carboxyvinyl polymers, water soluble polyacrylic acid/acrylate, or a combination thereof.

The water soluble polymer may be present in amounts of about 20 to about 95 wt %, specifically about 30 to about 85, and more specifically about 40 to about 75 wt % based on the total weight of the orally dissolving strip.

The orally dissolving strip can further optionally comprise a plasticizer in addition to the water soluble polymer and active agent. Exemplary plasticizers include propylene glycol, glycerin, glycerol, monoacetin, diacetin, triacetin, dimethyl phthalate, diethyl phthalate, dibutyl phthalate, dibutyl sebacate, triethyl titrate, tributyl citrate, triethyl citrate, triethyl acetyl citrate, castor oil, acetylated monoglycerides, sorbitol, or a combination thereof. The plasticizer may be present in amounts of about 0 to about 20, specifically about 1 to about 15, and more specifically about 5 to about 10 wt % based on the total weight of the orally dissolving strip.

The orally dissolving strip can further optionally comprise an emulsifying agent in addition to the water soluble polymer and active agent. Exemplary emulsifying agents include polyvinyl alcohol, a sorbitan esters, a cyclodextrin, benzyl benzoate, glyceryl monostearate, a polyoxyethylene alkyl ether, a polyoxyethylene stearate, poloxamer, a polyoxyethylene castor oil derivative, a hydrogenated vegetable oil, a polysorbate, or a combination thereof.

The emulsifying agent may be present in amounts of about 0 to about 20, specifically about 1 to about 15, and more specifically about 5 to about 10 wt % based on the total weight of the orally dissolving strip.

The orally dissolving strip can further optionally comprise a flavorant or sweetener in addition to the water soluble polymer and active agent. Exemplary sweeteners include sugar, a monosaccharide, an oligosaccharide, aldose, ketose, dextrose, maltose, lactose, glucose, fructose, sucrose, a sugar polyol (e.g., mannitol, xylitol, sorbitol, erythritol, and the like), artificial sweeteners (e.g., acesulfame potassium, sucralose, aspartame, saccharin, sodium saccharin, and the like) or a combination thereof. The sweetener may be present in amounts of about 0 to about 20, specifically about 1 to about 15, and more specifically about 5 to about 10 wt % based on the total weight of the orally dissolving strip.

In some embodiments, the orally dissolving formulations of the present invention may comprise an additional excipient. Suitable additional excipients include, but are not limited to, microcrystalline cellulose, colloidal silicon dioxide, talc, starch, or a combination thereof. Other optional components that can be used to prepare the orally dissolving strip include a filler/diluent, a surfactant, a disintegrating agent, an antifoaming agent, an antioxidant, a buffering agent, a colorant, or a combination thereof.

In one embodiment, the orally dissolving strip exhibits a drug loading of not more than 50% w/w of the film. Exemplary orally dissolving strips will comprise about 0.01 to about 50 mg of active agent per strip. In another embodiment, the orally dissolving strip has a thickness of about 0.1 to about 5.0 millimeters, specifically about 0.3 to about 4.0 and yet more specifically about 0.5 to about 2.5 millimeters. In another embodiment the orally dissolving strip has a surface area of about 1.0 to about 6.0, specifically about 1.2 to about 4.0 and yet more specifically about 1.5 to about 2.0 square centimeters.

The orally dissolving strip once placed in the oral cavity may dissolve after less than about 60 seconds, specifically less than 30 seconds, and yet more specifically less than about 20 seconds.

A solvent can be used in the process to prepare the orally dissolving strip, including water, ethanol, 1-butanol, 2-butanol, 2-ethoxyethanol, ethyl acetate, methyl acetate, 3-methyl-1-butanol, methylethyl ketone, 2-methyl-1-propanol, isobutyl acetate, isopropyl acetate ethyl ether, tert-butylmethyl ether acetone, or a combination thereof. The solvent is used for processing and then removed to result in the final product.

Methods of preparing orally dissolving strips involve solvent casting and film coating. The active agent is mixed with film-forming excipients and solvents such as water, ethanol, and the like. A thin coating of the mixture is cast on a moving, inert substrate and the coated substrate is moved through a drying oven to evaporate the solvent before die-cutting the dried film into strips. Another method involves hot-melt extrusion, by melting an active agent and excipient polymer blend which is then extruded through a die under molten conditions. The thin film is then cooled to room temperature and die-cut into strips.

In an embodiment, the PDE5 combination is formulated as an oral liquid dosage form (solution, suspension, dispersion, etc.), or in a reconstitutable form that when combined with a pharmaceutically acceptable oral liquid carrier results in an oral liquid dosage form. The liquid dosage forms generally include the PDE5 inhibitor, an additional active agent, and a pharmaceutically acceptable oral liquid carrier (alternatively “pharmaceutically acceptable oral liquid vehicle”). Additional optional ingredients include a suspending agent, a sweetener, a flavoring agent, a preservative, a pH adjusting agent, a colorant, or a combination thereof.

The PDE5 inhibitor and additional active agent can be present in the liquid composition in free form or in the form of a coated or uncoated granule, microtablet, pellet (as used herein “pellet” means a spherical granule prepared by extrusion and spheronization, and is equivalent to bead, spheroid, and microsphere), particle, or other multiparticulate system. The coating can include film forming coating, a taste-masking coating, a controlled-release coating, and the like.

Suitable liquid carriers include, for example, water; glycerin; propylene glycol; a lower polyethylene glycol (e.g., polyethylene glycol 200, polyethylene glycol 300, polyethylene glycol 400, polyethylene glycol 540, polyethylene glycol 600, and the like); ethanol; propylene carbonate; or a combination thereof.

The liquid carrier can be present in the liquid composition in an amount of about 30 to about 98 weight percent (wt %) based on the total weight of the liquid composition, specifically about 40 to about 90 wt %, more specifically about 50 to about 80 wt %, and yet more specifically about 60 to about 70 wt %.

The suspending agent for use in the liquid composition include, for example, a carbomer, a cellulose derivative such as powdered cellulose, methylcellulose, a hydroxyl alkyl cellulose such as hydroxyethyl cellulose, hydroxypropyl cellulose, or hydroxypropyl methylcellulose, carboxy methyl cellulose calcium, carboxy methyl cellulose sodium, polyvinylpyrrolidone; a natural gum such as gum acacia, carrageenan, sodium alginate, gellam gum, gum ghatti, guar gum, locust bean gum, tragacanth, xanthan gum; or a combination thereof.

A sweetener can be included in the liquid composition to make the composition palatable and more pleasing to the patient and to mask the taste of the active agents. Exemplary sweeteners include sugar alcohols (or polyols), such as glycerol, sorbitol, xylitol, mannitol, galactitol, maltitol, hydrogenated isomaltulose (isomalt), lactitol, erythritol, glucitol, ribitol, or a combination thereof; sugar sweeteners generally include saccharides, such as mono-saccharides, di-saccharides and poly-saccharides such as sucrose (saccharose, sugar), dextrose, maltose, dextrin, maltodextrin, xylose, ribose, glucose (including liquid glucose), mannose, galactose, fructose (levulose), lactose, invert sugar, fructo oligo saccharide syrups, trehalose, tagatos, fucose, gulose, raffinose, ribulose, rufinose, stachyose, xylulose, adonose, amylase, arabinose, deoxyribose, corn syrup solids, such as high fructose corn syrup, or a combination thereof; artificial sweeteners such as soluble saccharin salts, i.e., sodium or calcium saccharin salts, the potassium salt of 3,4-dihydro-6-methyl-1,2,3-oxathiazine-4-one-2,2-dioxide (Acesulfame-K), the free acid form of saccharin, L-aspartic acid derived sweeteners, such as L-aspartyl-L-phenylalanine methyl ester (Aspartame), L-alphaaspartyl-N-(2,2,4,4-tetramethyl-3-thietanyl)-D-alaninamide hydrate (Alitame), N-[N-(3,3-dimethylbutyl)-L-aspartyl]-L-phenylalanine 1-methyl ester (Neotame), methyl esters of L-aspartyl-L-phenylglycerine and L-aspartyl-L-2,5-dihydrophenyl-glycine, L-aspartyl-2,5-dihydro-L-phenylalanine; L-aspartyl-L-(1-cyclohexen)-alanine, or a combination thereof; maltol; or a combination thereof.

The sweetener can be present in the liquid composition in an amount of about 0.1 to about 75 wt % based on the total weight of the liquid composition, specifically about 5 to about 50 wt %, and more specifically about 2.5 to about 25 wt %. The amount of sweetener can be determined by one of ordinary skill in the art without undue experimentation. The use of sensory panels to determine the acceptable sweetness of the liquid composition may be used.

The liquid composition may optionally further comprise a flavoring agent. Flavoring agents include those flavors known to one of ordinary skill in the art, such as natural flavors and artificial flavors. Suitable amounts of flavoring agent can be selected by one of ordinary skill in the art without undue experimentation. In one embodiment, the flavoring agent can be present in the liquid composition from about 0.1 to about 8.0 wt % based on the total weight of the liquid composition, specifically about 0.4 to about 6 wt %, and more specifically about 1.0 to about 3.0 wt %.

In an embodiment, the liquid composition can further include a preservative to prevent the unwanted growth of bacteria, molds, fungi, or yeast. Examples of suitable preservatives include benzoic acid alkali metal salts (e.g., sodium benzoate), sorbic acid alkali metal salts (e.g., potassium sorbate), sodium erythorbate, sodium nitrite, calcium sorbate, butylated hydroxyanisole (BHA), butylated hydroxytoluene (BHT), parabens (e.g., lower alkyl esters of para-hydroxybenzoic acid), alkali metal salts of parabens including sodium and potassium salts of methyl-, ethyl-, propyl-, or butylparaben, or a combination thereof. Specific preservatives include sodium methylparaben, sodium propylparaben, and sodium butylparaben.

When used, the preservative can be present in the liquid composition in an amount of about 0.001 to about 0.3 wt % based on the total weight of the composition, specifically about 0.0075 to about 0.25 wt %, and yet more specifically about 0.01 to about 0.2 wt %.

In an embodiment, the oral liquid composition is preservative free.

The liquid composition optionally further comprises a colorant conventional in the pharmaceuticals art. Colorants can be used in amounts effective to produce a desired color for the composition. The colorants may include pigments, natural food colors, and dyes suitable for pharmaceutical applications.

The liquid composition optionally further includes a buffering agent or a pH adjusting agent to render the final liquid composition to a targeted pH. Suitable pH adjusting agents include pharmaceutically acceptable acids, bases, and their salts. Exemplary pH adjusting agents include alkali metal hydroxides (e.g., sodium hydroxide and potassium hydroxide), hydrochloric acid, alkali metal carbonates (e.g., sodium carbonate and potassium carbonate), carbonic acid, or a combination thereof. The pH adjusting agents can be used as solutions or suspensions in a pharmaceutically acceptable solvent. Suitable pharmaceutically acceptable solvents for use with the pH adjusting agent can include purified water, lower alkyl alcohols such as ethanol, a glycol, and the like, or a combination thereof.

The amount of pH adjusting agent can be any amount to result in a desired pH of the final liquid composition. Such amounts can be determined by one having ordinary skill in the art without undue experimentation.

In another embodiment, the combination of PDE5 inhibitor and an additional active agent is formulated in a powder form for single use, such as a sachet, to be suspended in a liquid carrier such as water or saliva. The powder form can be added to a glass of water with stirring or taken directly in the mouth where the ingredients are suspended in saliva and then swallowed.

In another embodiment, the combination of PDE5 inhibitor and an additional active agent is formulated in a powder form for multiple use, such as powder for suspensions in a bottle. A specified amount of water or a flavored solution can be added to constitute a suspension that is enough for multiple doses.

General components in the powder formulation include the active agents, a sweetener, and a suspending agent; optionally further comprising a flavorant, a colorant, a disintegrant, a combination thereof, and the like.

Also included herein are pharmaceutical kits comprising one multiple use, or a plurality of single use containers or units containing the PDE5 inhibitor combination dosage forms as described herein. The kits may further comprise one or more conventional pharmaceutical kit components, such as, for example, one or more containers to aid in facilitating compliance with a particular dosage regimen; one or more carriers; printed instructions, either as inserts or as labels, indicating quantities of the components to be administered, or guidelines for administration. Exemplary kits can be in the form of bubble or blister pack cards, optionally arranged in a desired order for a particular dosing regimen. Suitable blister packs can be arranged in a variety of configurations to accommodate a particular dosing regimen depending upon the additional active agent(s) used in the combination.

In the embodiment where the PDE5 inhibitor and the additional active agent(s) are formulated as separate dosage units, the dosage units can be packaged in a bubble or blister pack cards comprising each of the active agents to be used in the combination, optionally arranged in the pack in easily divided dosing units comprising each of the active agents. For example, a single blister pack card can comprise five tablets of a PDE5 inhibitor and five tablets of extended release acetaminophen, each of the tablets arranged in the blister pack card as five tear-away blister pack units, each comprising one tablet of PDE5 inhibitor and one tablet of extended release acetaminophen. The patient can carry the entire blister pack or the patient can tear-away one or more of the blister pack units and keep on hand until the time of dosing.

Those forms existing as liquids (e.g., solution, emulsion, or suspension) can be packaged for convenient dosing in prepackaged, single use containers, or in containers comprising multiple doses.

A “patient” as used herein means a human in need of medical treatment. Medical treatment can include treatment of an existing condition, such as a disease or disorder, prophylactic or preventative treatment, or diagnostic treatment.

A method of treating a condition treatable with PDE5 inhibitor therapy comprises administering a PDE5 inhibitor and an additional active agent to a patient in need thereof, wherein the additional active agent is an analgesic, an antacid, an anti-migraine agent, a H2 blocker, a proton pump inhibitor, an anti-gas agent, or a combination thereof, and further wherein the combination treatment results in a reduction or elimination of a side effect associated with PDE5 inhibitor therapy alone. Within this embodiment, the side effects that are reduced or eliminated include headaches, migraines, back pain, myalgia, dyspepsia, gastritis, heartburn, or a combination thereof. Within these embodiments, the condition to be treated is erectile dysfunction, benign prostatic hyperplasia, idiopathic pulmonary hypertension/pulmonary arterial hypertension (“PAH”), premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, a combination thereof, or any other condition that is treated with PDE5 inhibitors.

In an embodiment, a method of orally administering a PDE5 inhibitor combination comprises concomitantly administering orally to a patient in need of PDE5 treatment (e.g., for ED, BPH, PAH) a therapeutically effective amount of a PDE5 inhibitor and a therapeutically effective amount of an additional active agent that reduces or eliminates a side effect typically caused by PDE5 inhibitor therapy (e.g., headache, migraine, back pain, myalgia, dyspepsia, gastritis, heartburn, or a combination thereof), the additional active agent is an analgesic (specifically a NSAID), an antacid, an anti-migraine agent, a H2 blocker, a proton pump inhibitor, an anti-gas agent, or a combination thereof. As used herein, “concomitantly administering” means the PDE5 inhibitor and the additional active agent are administered simultaneously, or within fifteen minutes of one another, specifically within ten minutes, and more specifically within five minutes of one another. In an embodiment, the PDE5 inhibitor and the additional active agent are in the same dosage form. In an embodiment, the PDE5 inhibitor and the additional active agent are in separate dosage forms, but packaged together to facilitate concomitant administration.

In an embodiment, a method of orally administering a PDE5 inhibitor combination comprises concomitantly administering orally to a patient in need of PDE5 treatment (e.g., for ED, BPH, PAH) a therapeutically effective amount of a PDE5 inhibitor and a therapeutically effective amount of an analgesic (specifically a NSAID), wherein a side effect of headache, migraine, back pain, myalgia, or a combination thereof is substantially reduced or eliminated.

In an embodiment, a method of orally administering a PDE5 inhibitor combination comprises concomitantly administering orally to a patient in need of PDE5 treatment (e.g., for ED, BPH, PAH) a therapeutically effective amount of a PDE5 inhibitor and a therapeutically effective amount of an antacid, a H2 blocker, a proton pump inhibitor, an anti-gas agent, or a combination thereof, wherein a side effect of dyspepsia, gastritis, heartburn, or a combination thereof is substantially reduced or eliminated.

In an embodiment, a method of orally administering a PDE5 inhibitor combination comprises concomitantly administering orally to a patient in need of PDE5 treatment (e.g., for ED, BPH, PAH) a therapeutically effective amount of a PDE5 inhibitor and a therapeutically effective amount of anti-migraine agent, wherein a side effect of headache, migraine, or a combination thereof is substantially reduced or eliminated.

In an embodiment, a method of orally administering a PDE5 inhibitor combination comprises concomitantly administering orally to a patient in need of PDE5 treatment (e.g., for ED, BPH, PAH) a therapeutically effective amount of a PDE5 inhibitor, a therapeutically effective amount of an analgesic (specifically a NSAID), and a therapeutically effective amount of an antacid, an anti-migraine agent, a H2 blocker, a proton pump inhibitor, an anti-gas agent, or a combination thereof, wherein a side effect of headache, migraine, back pain, myalgia, dyspepsia, gastritis, heartburn, or a combination thereof is substantially reduced or eliminated.

The following examples are merely illustrative of the compositions and methods disclosed herein and are not intended to limit the scope hereof.

### EXAMPLES

### Example 1 Preparation of Sildenafil Citrate 50 mg and Naproxen Sodium, 200 mg Immediate Release Tablet Formulation

A sildenafil citrate and naproxen sodium compressed, immediate release tablet formulation is prepared from the ingredients in Table 1.

TABLE 1

| Component | Mg/Tablet |
|---|---|
| Base granules | |
| Sildenafil Citrate | 50 |
| Naproxen Sodium | 220 |
| Polyvinylpyrrolidone (PLASDONE K29/32) | 7 |
| SD3A Alcohol | * |
| Compression Mix | |
| Base granules | 227 |
| Microcrystalline cellulose (AVICEL PH 101) | 128 |
| Hydroxy propylcellulose (KLUCEL EXF) | 26 |
| Crosslinked PVP (POLYPLASDONE XL) | 16 |
| Magnesium Stearate | 3 |
| Total | 450 |

* not in final formulation

Sildenafil citrate and naproxen sodium are weighed. PLASDONE K29/32 is dissolved in SD3A alcohol. Sildenafil Citrate and Naproxen Sodium are then granulated with the PLASDONE K29/32 solution. The granules are then dried in an oven at 45° C. and milled using a suitable mill. The AVICEL PH 101, KLUCEL EXF and POLYPLASDONE XL are passed through a #20 mesh screen. The screened materials are then blended with the milled sildenafil citrate and naproxen sodium granules. The magnesium stearate is passed through a #30 mesh screen. The screened magnesium stearate is then added to the above blend and mixed well. 450 mg of the blend is compressed to make sildenafil citrate and naproxen sodium immediate release tablets comprising 50 mg of sildenafil citrate and 200 mg of naproxen.

### Example 2 Preparation of Sildenafil Citrate 50 mg and Acetaminophen, 325 mg Immediate Release Tablet Formulation

A sildenafil citrate and acetaminophen compressed, immediate release tablet formulation is prepared from the ingredients in Table 2.

TABLE 2

| Component | Mg/Tablet |
|---|---|
| Base granules | |
| Sildenafil Citrate | 50 |
| Acetaminophen | 325 |
| Polyvinylpyrrolidone (PLASDONE K29/32) | 5 |
| Purified water | * |
| Compression Mix | |
| Base granules | 380 |
| Microcrystalline cellulose (AVICEL PH 101) | 50 |
| Hydroxy propylcellulose (KLUCEL EXF) | 5 |
| Lactose monohydrate | 58 |
| Croscarmellose Sodium (AC-DI-SOL) | 6 |
| Magnesium Stearate | 1 |
| Total | 500 |

* not in final formulation

Sildenafil citrate and acetaminophen are weighed. Plasdone K29/32 is dissolved in purified water. Sildenafil citrate and acetaminophen are granulated with the PLASDONE K29/32 solution. The granules are dried in an oven at 50° C. and milled using a suitable mill. AVICEL PH 101, KLUCEL EXF, lactose monohydrate and AC-DI-SOL are passed through a #20 mesh screen. The screened materials are then blended with milled sildenafil citrate and acetaminophen granules. Magnesium stearate is passed through a #30 mesh screen. The screened magnesium stearate is added to the above blend and mixed well. 500 mg of the blend is compressed to make sildenafil citrate 50 mg and acetaminophen 325 mg immediate release tablets.

### Example 3 Preparation of Tadalafil 10 mg and Celecoxib, 100 mg Immediate Release Capsule Formulation

A tadalafil and celecoxib immediate release capsule formulation is prepared from the ingredients in Table 3.

TABLE 3

| Component | Mg/Capsule |
|---|---|
| Base granules | |
| Tadalafil | 10 |
| Celecoxib | 100 |
| Polyvinylpyrrolidone (PLASDONE K29/32) | 8 |
| Purified water | * |
| Encapsulation Mix | |
| Base granules | 118 |
| Microcrystalline cellulose (AVICEL PH 101) | 65 |
| Lactose monohydrate | 70 |
| Croscarmellose Sodium (AC-DI-SOL) | 6 |
| Magnesium Stearate | 1 |
| Total | 260 |

* not in final formulation

Tadalafil and celecoxib are weighed. PLASDONE K29/32 is dissolved in purified water. Tadalafil and celecoxib are granulated with the PLASDONE K29/32 solution. The granules are dried in an oven at 50° C. and milled using a suitable mill. AVICEL PH 101, Lactose monohydrate and AC-DI-SOL are passed through a #20 mesh screen. The screened materials are then blended with milled tadalafil and celecoxib granules. Magnesium stearate is passed through a #30 mesh screen. The screened magnesium stearate is added to the above blend and mixed well. 260 mg of the blend is encapsulated to make tadalafil 10 mg and celecoxib 100 mg immediate release capsules.

### Example 4 Preparation of Naproxen Sodium, 300 mg Extended Release Tablet Formulation

A Naproxen Sodium compressed, extended release tablet formulation is prepared from the ingredients in Table 4.

TABLE 4

| Component | Mg/Tablet |
|---|---|
| Base granules | |
| Naproxen Sodium | 330 |
| Carnauba wax | 210 |
| Ethyl cellulose powder | 50 |
| Stearic acid | 50 |
| SD3A Alcohol | * |
| Compression Mix | |
| Base granules | 640 |
| Silicon Dioxide (SYLOID 244 FP) | 5 |
| Magnesium Stearate | 5 |
| Total | 650 |

* not in final formulation

Naproxen sodium, carnauba wax and ethyl cellulose powder are mixed. Stearic acid is dissolved in SD3A alcohol by heating the alcohol to 50° C. The powder mix is granulated with stearic acid solution. The granules are dried in an oven at 35° C. and milled using a suitable mill. SYLOID is passed through a #20 mesh screen. The milled granules and screened SYLOID are blended in a blender. Magnesium stearate is passed through a #30 mesh screen. The screened magnesium stearate is added to the blender and mixed well. 650 mg of the blend is compressed to make naproxen sodium extended release tablets comprising 300 mg of naproxen.

### Example 5 Preparation of Acetaminophen, 325 mg Extended Release Tablet Formulation

An acetaminophen compressed, extended release tablet formulation is prepared from the ingredients in Table 5.

TABLE 5

| Component | Mg/Tablet |
|---|---|
| Base granules | |
| Acetaminophen | 325 |
| Carnauba wax | 150 |
| Ethyl cellulose powder | 40 |
| Stearic acid | 50 |
| SD3A Alcohol | * |
| Compression Mix | |
| Base granules | 565 |
| Silicon Dioxide (SYLOID 244 FP) | 5 |
| Magnesium Stearate | 5 |
| Total | 575 |

* not in final formulation

Acetaminophen, carnauba wax and ethyl cellulose powder are mixed. Stearic acid is dissolved in SD3A alcohol by heating the alcohol to 50° C. The powder mix is granulated with stearic acid solution. The granules are dried in an oven at 35° C. and milled using a suitable mill. SYLOID is passed through a #20 mesh screen. The milled granules and screened SYLOID are blended in a blender. Magnesium stearate is passed through a #30 mesh screen. The screened magnesium stearate is added to the blender and mixed well. 575 mg of the blend is compressed to make acetaminophen, 325 mg extended release tablet.

### Example 6 Preparation of Sildenafil Citrate 100 mg and Naproxen Sodium, 500 mg Bi-Layer Immediate/Extended Release Tablets

A sildenafil and naproxen sodium immediate release portion of the tablet formulation is prepared from the ingredients in Table 6.

TABLE 6

| Component | Mg/Tablet |
|---|---|
| Base granules | |
| Sildenafil Citrate | 100 |
| Naproxen Sodium | 220 |
| Polyvinylpyrrolidone (PLASDONE K29/32) | 7 |
| SD3A Alcohol | * |
| Compression Mix | |
| Base granules | 227 |
| Microcrystalline cellulose (AVICEL PH 101) | 128 |
| Hydroxy propylcellulose (KLUCEL EXF) | 26 |
| Crosslinked PVP (POLYPLASDONE XL) | 16 |
| Magnesium Stearate | 3 |
| Total | 500 |

* not in final formulation

Sildenafil citrate and naproxen sodium are weighed. PLASDONE K29/32 is dissolved in SD3A alcohol. Sildenafil citrate and naproxen sodium are then granulated with the PLASDONE K29/32 solution. The granules are then dried in an oven at 45° C. and milled using a suitable mill. The AVICEL PH 101, KLUCEL EXF and POLYPLASDONE XL are passed through a #20 mesh screen. The screened materials are then blended with the milled sildenafil citrate and naproxen sodium granules. The magnesium stearate is passed through a #30 mesh screen. The screened magnesium stearate is then added to the above blend and mixed well.

500 mg of the blend and the extended release formulation prepared according to Example 4 are then compressed into bi-layer tablets using an alternative tablet press. Each tablet contains 100 mg of sildenafil citrate and 500 mg of naproxen.

### Example 7 Bi-Layer Immediate/Extended Release Tablets Comprising 50 mg of Sildenafil Citrate and 650 mg of Acetaminophen

An immediate release formulation is prepared by wet granulation according to Example 2, and an extended release formulation is prepared by wet granulation according to Example 5. The mixtures are then compressed into bi-layer tablets using an alternative tablet press. Each tablet contains 50 mg of sildenafil citrate and 650 mg acetaminophen.

### Example 8 Preparation of Tadalafil 10 mg and Esomeprazole 20 mg Capsule Formulation

A tadalafil and esomeprazole capsule formulation is prepared from the ingredients in Table 7.

TABLE 7

| Component | Mg/Capsule |
|---|---|
| Tadalafil | 10 |
| Lactose monohydrate | 80 |
| Microcrystalline cellulose (AVICEL PH 101) | 70 |
| Polyvinylpyrrolidone (PLASDONE K29/32) | 8 |
| Purified water | * |
| Croscarmellose Sodium (AC-DI-SOL) | 6 |
| Magnesium Stearate | 1 |
| Esomeprazole Enteric coated granules | 75 |
| (equivalent to 20 mg of Esomeprazole) | |
| Total | 250 |

* not in final formulation

Tadalafil, AVICEL PH 101 and lactose monohydrate are weighed. PLASDONE K29/32 is dissolved in purified water. Tadalafil, AVICEL PH 101 and Lactose monohydrate are granulated with the PLASDONE K29/32 solution. The granules are dried in an oven at 50° C. and milled using a suitable mill. AC-DI-SOL is passed through a #20 mesh screen. The screened materials are then blended with milled tadalafil granules. Esomeprazole enteric coated granules (equivalent to 20 mg of esomeprazole) are added to tadalafil granules and blended. Magnesium stearate is passed through a #30 mesh screen. The screened magnesium stearate is added to the above blend and mixed well. 250 mg of the blend is encapsulated to make tadalafil 10 mg and esomeprazole 20 mg capsules.

### Example 9 Preparation of Sildenafil Citrate 50 mg and Esomeprazole 20 mg Powder for Suspension

A sildenafil citrate and esomeprazole powder for suspension formulation is prepared from the ingredients in Table 8.

TABLE 8

| Component | Mg/Dose |
|---|---|
| Sildenafil Citrate | 50 |
| Sorbitol Powder | 500 |
| Sucralose | 3 |
| Sodium Benzoate | 2 |
| Xanthan Gum | 7 |
| Sodium Citrate | 3 |
| Citric Acid | 4 |
| Silicon Dioxide | 2 |
| Titanium Dioxide | 3 |
| Grape Flavor | 1 |
| Esomeprazole Enteric coated granules | 75 |
| (equivalent to 20 mg of Esomeprazole) | |
| Total | 650 |

Sildenafil citrate and sorbitol powder are passed through a #20 mesh screen. The screened materials are then blended. Sucralose, sodium benzoate, xanthan gum, sodium citrate, citric acid, silicon dioxide and grape flavor are passed through a #20 mesh screen. The screened materials are added to the blender containing sildenafil citrate and sorbitol powder and blended thoroughly. Esomeprazole enteric coated granules (equivalent to 20 mg of esomeprazole) are added to the blender and mixed well. 650 mg of the blend is encapsulated into packets.

### Example 10 Bi-Layer Immediate/Extended Release Tablets Comprising 10 mg of Tadalafil, 20 Mg of Esomeprazole and 500 mg of Naproxen

A bi-layer immediate/extended release tablet comprising 10 mg of tadalafil, 20 mg of esomeprazole and 500 mg of naproxen formulation is prepared from the ingredients in Table 9.

TABLE 9

| Component | Mg/Tablet |
|---|---|
| Tadalafil | 10 |
| Naproxen Sodium | 220 |
| Lactose monohydrate | 80 |
| Microcrystalline cellulose (AVICEL PH 101) | 70 |
| Polyvinylpyrrolidone (PLASDONE K29/32) | 8 |
| Purified water | * |
| Croscarmellose Sodium (AC-DI-SOL) | 6 |
| Magnesium Stearate | 1 |
| Esomeprazole Enteric coated granules | 75 |
| (equivalent to 20 mg of Esomeprazole) | |
| Total | 470 |

Tadalafil, naproxen sodium, AVICEL PH 101 and lactose monohydrate are weighed. PLASDONE K29/32 is dissolved in purified water. Tadalafil, Napeoxen Sodium, AVICEL PH 101 and lactose monohydrate are granulated with the PLASDONE K29/32 solution. The granules are dried in an oven at 50° C. and milled using a suitable mill. AC-DI-SOL is passed through a #20 mesh screen. The screened materials are then blended with milled tadalafil and naproxen granules. Esomeprazole enteric coated granules (equivalent to 20 mg of esomeprazole) are added to tadalafil and naproxen granules and blended. Magnesium stearate is passed through a #30 mesh screen. The screened magnesium stearate is added to the above blend and mixed well. 470 mg of the final blend is compressed with 650 mg of the blend prepared according to Example 4 into bi-layer tablets using an alternative tablet press. Each tablet contains 10 mg of Tadalafil, 20 mg of esomeprazole and 500 mg of naproxen.

### Example 11 Preparation of Tadalafil 10 mg Immediate Release and Propranolol HCl 80 mg Extended Release Capsule Formulation

A tadalafil and propranolol HCl capsule formulation is prepared from the ingredients in Table 10.

TABLE 10

| Component | Mg/Capsule |
|---|---|
| Tadalafil | 10 |
| Lactose monohydrate | 80 |
| Microcrystalline cellulose (AVICEL PH 101) | 70 |
| Polyvinylpyrrolidone (PLASDONE K29/32) | 8 |
| Purified water | * |
| Croscarmellose Sodium (AC-DI-SOL) | 6 |
| Magnesium Stearate | 1 |
| Propranolol HCI Extended Release pellets | 200 |
| (equivalent to 80 mg of propranolol HCl) | |
| Total | 375 |

* not in final formulation

Tadalafil, AVICEL PH 101 and lactose monohydrate are weighed. PLASDONE K29/32 is dissolved in purified water. Tadalafil, AVICEL PH 101 and lactose monohydrate are granulated with the PLASDONE K29/32 solution. The granules are dried in an oven at 50° C. and milled using a suitable mill. AC-DI-SOL is passed through a #20 mesh screen. The screened materials are then blended with milled tadalafil granules. Propranolol HCl extended release pellets (equivalent to 80 mg of propranolol HCl) are added to tadalafil granules and blended. Magnesium stearate is passed through a #30 mesh screen. The screened magnesium stearate is added to the above blend and mixed well. 375 mg of the blend is encapsulated to make tadalafil 10 mg immediate release and propranolol HCl 80 mg extended release capsules.

The compositions and methods disclosed herein include(s) at least the following aspects:

Aspect 1: A combination oral formulation, comprising a phosphodiesterase-5 inhibitor; and an additional active agent that can reduce or eliminate a side effect associated with phosphodiesterase-5 inhibitor, wherein the additional active agent is an analgesic, an antacid, an anti-migraine agent, a H2 blocker, a proton pump inhibitor, an anti-gas agent, or a combination thereof.

Aspect 2: The formulation of Aspect 1, wherein the phosphodiesterase-5 inhibitor is avanafil, sildenafil, tadalafil, vardenafil, lodenafil, udenafil, mirodenafil, a pharmaceutically acceptable salt thereof, or a combination thereof, specifically the phosphodiesterase-5 inhibitor is avanafil, sildenafil citrate, tadalafil, vardenafil monohydrochloride, or a combination thereof; the analgesic is a non-steroidal, anti-inflammatory or a pharmaceutically acceptable salt thereof, specifically the analgesic is acemetacin, acetaminophen, aminoprofen, aspirin, benoxaprofen, bucloxic acid, carisoprodol, carprofen, celecoxib, clidanac, cyclobenzaprine, diclofenac, diflurisal, fentiazac, flufenamic acid, flurbiprofen, fenoprofen, flubufen, flufenisal, isoxicam, ketoprofen, ibuprofen, indomethacin, indoprofen, ketorolac, naproxen, nabumetone, meclofenamic acid, meclofenate, mefenamic acid, metaxalone, methocarbamol, muroprofen, niflumic acid, orphenadrine, oxaprozin, oxpinac, piroprofen, piroxicam, pramoprofen, sudoxicam, sulindac, suprofen, tiaprofenic acid, tiopinac, tolmetin, tolfenamic acid, trioxaprofen, zidometacin, zomepirac, or a pharmaceutically acceptable salt thereof, and more specifically the analgesic is naproxen sodium, orphenadrine citrate, or a combination thereof; the antacid is a pharmaceutically acceptable alkali or alkaline earth metal carbonate, a pharmaceutically acceptable alkali or alkaline earth metal hydroxide, or a combination thereof, specifically calcium carbonate, magnesium carbonate, sodium bicarbonate, aluminum hydroxide, magnesium hydroxide, or a combination thereof; the anti-migraine agent is a beta blocker, a calcium channel blocker, an antidepressant, an antiseizure medication, a CGRP inhibitor, a pharmaceutically acceptable salt thereof, or a combination thereof, specifically the beta blocker is acebutolol, atenolol, betaxolol, bisoprolol, metoprolol, nadolol, propranolol, timolol, a pharmaceutically acceptable salt thereof, or a combination thereof, the calcium channel blocker is nimodipine, verapamil, a pharmaceutically acceptable salt thereof, or a combination thereof, the antidepressant is amitriptyline, nortriptyline, a pharmaceutically acceptable salt thereof, or a combination thereof, the antiseizure medication is gabapentin, topiramate, valproic acid, a pharmaceutically acceptable salt thereof, or a combination thereof, and the CGRP inhibitor is erenumab, fremanezumab, a pharmaceutically acceptable salt thereof, or a combination thereof; the H2 blocker is cimetidine, famotidine, nizatidine, ranitidine, a pharmaceutically acceptable salt thereof, or a combination thereof, specifically the H2 blocker is cimetidine hydrochloride, famotidine, nizatidine, ranitidine hydrochloride, or a combination thereof; the proton pump inhibitor is esomeprazole, dexlansoprazole, lansoprazole, omeprazole, pantoprazole, rabeprazole, a pharmaceutically acceptable salt thereof, or a combination thereof, specifically the proton pump inhibitor is esomeprazole magnesium, esomeprazole sodium, esomeprazole strontium, dexlansoprazole, lansoprazole, omeprazole magnesium, omeprazole sodium, omeprazole strontium, pantoprazole sodium, rabeprazole sodium, or a combination thereof; and the anti-gas agent is simethicone.

Aspect 3: The formulation of Aspect 1 or 2, wherein each of the phosphodiesterase-5 inhibitor and the additional active agent individually are formulated for immediate release or controlled release.

Aspect 4: The formulation of Aspect 3, wherein the controlled release is achieved with a controlled release matrix, a functional coating, or a combination thereof; and wherein the controlled release is extended release, delayed release, pulsed release, or a combination thereof.

Aspect 5: The formulation of any one of Aspects 1-4, wherein the oral formulation is a solid, semisolid, or liquid formulation; specifically wherein the oral formulation is a tablet, a capsule, a pellet, a film, a sachet, a powder, a chewable gummy, a solution, a suspension, or an emulsion.

Aspect 6: The formulation of any one of Aspects 1-5, wherein the oral formulation is a compressed tablet or a capsule.

Aspect 7: The formulation of any one of Aspects 1-2, wherein

the oral formulation is an immediate release tablet or capsule formulation comprising a combination of sildenafil citrate or tadalafil as the phosphodiesterase-5 inhibitor and naproxen sodium, acetaminophen, ibuprofen, or celecoxib as the analgesic for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of headache, migraine, back pain, myalgia, or a combination thereof; the oral formulation is a tablet or a capsule formulation comprising a combination of immediate release sildenafil citrate or tadalafil as the phosphodiesterase-5 inhibitor and extended release naproxen sodium, acetaminophen, ibuprofen, or celecoxib as the analgesic for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of headache, migraine, back pain, myalgia, or a combination thereof; the oral formulation is a tablet or a capsule formulation comprising a combination of immediate release sildenafil citrate or tadalafil as the phosphodiesterase-5 inhibitor and enteric coated esomeprazole subunits as the proton pump inhibitor for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of dyspepsia, gastritis, heartburn, or a combination thereof, wherein the subunits are a plurality of granules, microtablets, minitablets, caplets, pellets, or particles; the oral formulation is a tablet or a capsule formulation comprising a combination of immediate release sildenafil citrate or tadalafil as the phosphodiesterase-5 inhibitor and extended release propranolol HCl subunits as the beta blocker for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of migraine, wherein the subunits are a plurality of granules, microtablets, minitablets, caplets, pellets, or particles; the oral formulation is a tablet or a capsule formulation comprising a combination of immediate release sildenafil citrate or tadalafil as the phosphodiesterase-5 inhibitor, immediate release and/or extended release naproxen sodium, acetaminophen, ibuprofen, or celecoxib as the analgesic, and enteric coated esomeprazole subunits as the proton pump inhibitor for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of headache, migraine, back pain, myalgia, dyspepsia, gastritis, heartburn, or a combination thereof, and wherein the subunits are a plurality of granules, microtablets, minitablets, caplets, pellets, or particles; the oral formulation is a tablet or a capsule formulation comprising a combination of immediate release sildenafil citrate or tadalafil as the phosphodiesterase-5 inhibitor and immediate release ranitidine hydrochloride as the H2 blocker for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of dyspepsia, gastritis, heartburn, or a combination thereof; the oral formulation is a tablet or a capsule formulation comprising a combination of immediate release sildenafil citrate or tadalafil as the phosphodiesterase-5 inhibitor and immediate release calcium carbonate, magnesium carbonate, sodium bicarbonate, aluminum hydroxide, or magnesium hydroxide as the antacid for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of dyspepsia, gastritis, heartburn, or a combination thereof; the oral formulation is a powder for suspension comprising a combination of immediate release sildenafil citrate or tadalafil as the phosphodiesterase-5 inhibitor and enteric coated esomeprazole granules as the proton pump inhibitor for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of dyspepsia, gastritis, heartburn, or a combination thereof; the oral formulation is a powder for suspension comprising a combination of immediate release sildenafil citrate or tadalafil as the phosphodiesterase-5 inhibitor and an extended release Naproxen Sodium, acetaminophen, ibuprofen, or celecoxib pellets as the analgesic for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of headache, migraine, back pain, myalgia, or a combination thereof; the oral formulation is a powder for suspension comprising a combination of immediate release sildenafil citrate or tadalafil as the phosphodiesterase-5 inhibitor and extended release propranolol HCl subunits as the beta blocker for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of migraine, wherein the subunits are a plurality of granules, microtablets, minitablets, caplets, pellets, or particles.

Aspect 8: A kit comprising the formulation of any one of Aspects 1-7.

Aspect 9: A method of treating a condition treatable with phosphodiesterase-5 inhibitor therapy, comprising administering a combination oral formulation comprising a phosphodiesterase-5 inhibitor and an additional active agent to a patient in need of phosphodiesterase-5 inhibitor therapy thereof; wherein the additional active agent is an analgesic, an antacid, an anti-migraine agent, a H2 blocker, a proton pump inhibitor, an anti-gas agent, or a combination thereof; and wherein the combination results in a reduction or elimination of a side effect associated with phosphodiesterase-5 inhibitor therapy alone.

Aspect 10: The method of Aspect 9, wherein the condition to be treated is erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, and wherein the side effect is headache, migraine, back ache, myalgia, dyspepsia, gastritis, heartburn, or a combination thereof.

Aspect 11: The method of Aspect 9 or 10, wherein the phosphodiesterase-5 inhibitor is avanafil, sildenafil, tadalafil, vardenafil, lodenafil, udenafil, mirodenafil, a pharmaceutically acceptable salt thereof, or a combination thereof, specifically the phosphodiesterase-5 inhibitor is avanafil, sildenafil citrate, tadalafil, vardenafil monohydrochloride, or a combination thereof; the analgesic is a non-steroidal, anti-inflammatory or a pharmaceutically acceptable salt thereof, specifically the analgesic is acemetacin, acetaminophen, aminoprofen, aspirin, benoxaprofen, bucloxic acid, carisoprodol, carprofen, celecoxib, clidanac, cyclobenzaprine, diclofenac, diflurisal, fentiazac, flufenamic acid, flurbiprofen, fenoprofen, flubufen, flufenisal, isoxicam, ketoprofen, ibuprofen, indomethacin, indoprofen, ketorolac, naproxen, nabumetone, meclofenamic acid, meclofenate, mefenamic acid, metaxalone, methocarbamol, muroprofen, niflumic acid, orphenadrine, oxaprozin, oxpinac, piroprofen, piroxicam, pramoprofen, sudoxicam, sulindac, suprofen, tiaprofenic acid, tiopinac, tolmetin, tolfenamic acid, trioxaprofen, zidometacin, zomepirac, or a pharmaceutically acceptable salt thereof, and more specifically the analgesic is naproxen sodium, orphenadrine citrate, or a combination thereof; the antacid is a pharmaceutically acceptable alkali or alkaline earth metal carbonate, a pharmaceutically acceptable alkali or alkaline earth metal hydroxide, or a combination thereof, specifically calcium carbonate, magnesium carbonate, sodium bicarbonate, aluminum hydroxide, magnesium hydroxide, or a combination thereof; the anti-migraine agent is a beta blocker, a calcium channel blocker, an antidepressant, an antiseizure medication, a CGRP inhibitor, a pharmaceutically acceptable salt thereof, or a combination thereof, specifically the beta blocker is acebutolol, atenolol, betaxolol, bisoprolol, metoprolol, nadolol, propranolol, timolol, a pharmaceutically acceptable salt thereof, or a combination thereof, the calcium channel blocker is nimodipine, verapamil, a pharmaceutically acceptable salt thereof, or a combination thereof, the antidepressant is amitriptyline, nortriptyline, a pharmaceutically acceptable salt thereof, or a combination thereof, the antiseizure medication is gabapentin, topiramate, valproic acid, a pharmaceutically acceptable salt thereof, or a combination thereof, and the CGRP inhibitor is erenumab, fremanezumab, a pharmaceutically acceptable salt thereof, or a combination thereof; the H2 blocker is cimetidine, famotidine, nizatidine, ranitidine, a pharmaceutically acceptable salt thereof, or a combination thereof, specifically the H2 blocker is cimetidine hydrochloride, famotidine, nizatidine, ranitidine hydrochloride, or a combination thereof; the proton pump inhibitor is esomeprazole, dexlansoprazole, lansoprazole, omeprazole, pantoprazole, rabeprazole, a pharmaceutically acceptable salt thereof, or a combination thereof, specifically the proton pump inhibitor is esomeprazole magnesium, esomeprazole sodium, esomeprazole strontium, dexlansoprazole, lansoprazole, omeprazole magnesium, omeprazole sodium, omeprazole strontium, pantoprazole sodium, rabeprazole sodium, or a combination thereof; and the anti-gas agent is simethicone.

Aspect 12: The method of any one of Aspects 9-11, wherein each of the phosphodiesterase-5 inhibitor and the additional active agent individually are formulated for immediate release or controlled release.

Aspect 13. The method of Aspect 12, wherein the controlled release is achieved with a controlled release matrix, a functional coating, or a combination thereof; and wherein the controlled release is extended release, delayed release, pulsed release, or a combination thereof.

Aspect 14: The method of any one of Aspects 9-13, wherein the oral formulation is a solid, semisolid, or liquid formulation; specifically wherein the oral formulation is a tablet, a capsule, a pellet, a film, a sachet, a powder, a chewable gummy, a solution, a suspension, or an emulsion.

Aspect 15: The method of any one of Aspects 9-14, wherein the oral formulation is a compressed tablet or a capsule.

Aspect 16: The method of any one of Aspects 9-11, wherein

the oral formulation is an immediate release tablet or capsule formulation comprising a combination of sildenafil citrate or tadalafil as the phosphodiesterase-5 inhibitor and naproxen sodium, acetaminophen, ibuprofen, or celecoxib as the analgesic for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of headache, migraine, back pain, myalgia, or a combination thereof; the oral formulation is a tablet or a capsule formulation comprising a combination of immediate release sildenafil citrate or tadalafil as the phosphodiesterase-5 inhibitor and extended release naproxen sodium, acetaminophen, ibuprofen, or celecoxib as the analgesic for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of headache, migraine, back pain, myalgia, or a combination thereof; the oral formulation is a tablet or a capsule formulation comprising a combination of immediate release sildenafil citrate or tadalafil as the phosphodiesterase-5 inhibitor and enteric coated esomeprazole subunits as the proton pump inhibitor for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of dyspepsia, gastritis, heartburn, or a combination thereof, wherein the subunits are a plurality of granules, microtablets, minitablets, caplets, pellets, or particles; the oral formulation is a tablet or a capsule formulation comprising a combination of immediate release sildenafil citrate or tadalafil as the phosphodiesterase-5 inhibitor and extended release propranolol HCl subunits as the beta blocker for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of migraine, wherein the subunits are a plurality of granules, microtablets, minitablets, caplets, pellets, or particles; the oral formulation is a tablet or a capsule formulation comprising a combination of immediate release sildenafil citrate or tadalafil as the phosphodiesterase-5 inhibitor, immediate release and/or extended release naproxen sodium, acetaminophen, ibuprofen, or celecoxib as the analgesic, and enteric coated esomeprazole subunits as the proton pump inhibitor for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of headache, migraine, back pain, myalgia, dyspepsia, gastritis, heartburn, or a combination thereof, and wherein the subunits are a plurality of granules, microtablets, minitablets, caplets, pellets, or particles; the oral formulation is a tablet or a capsule formulation comprising a combination of immediate release sildenafil citrate or tadalafil as the phosphodiesterase-5 inhibitor and immediate release ranitidine hydrochloride as the H2 blocker for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of dyspepsia, gastritis, heartburn, or a combination thereof; the oral formulation is a tablet or a capsule formulation comprising a combination of immediate release sildenafil citrate or tadalafil as the phosphodiesterase-5 inhibitor and immediate release calcium carbonate, magnesium carbonate, sodium bicarbonate, aluminum hydroxide, or magnesium hydroxide as the antacid for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of dyspepsia, gastritis, heartburn, or a combination thereof; the oral formulation is a powder for suspension comprising a combination of immediate release sildenafil citrate or tadalafil as the phosphodiesterase-5 inhibitor and enteric coated esomeprazole granules as the proton pump inhibitor for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of dyspepsia, gastritis, heartburn, or a combination thereof; the oral formulation is a powder for suspension comprising a combination of immediate release sildenafil citrate or tadalafil as the phosphodiesterase-5 inhibitor and an extended release Naproxen Sodium, acetaminophen, ibuprofen, or celecoxib pellets as the analgesic for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of headache, migraine, back pain, myalgia, or a combination thereof; or the oral formulation is a powder for suspension comprising a combination of immediate release sildenafil citrate or tadalafil as the phosphodiesterase-5 inhibitor and extended release propranolol HCl subunits as the beta blocker for the treatment of erectile dysfunction, benign prostatic hyperplasia, idiopathic pulmonary hypertension, premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof, wherein the patient experiences a reduction or elimination of migraine, wherein the subunits are a plurality of granules, microtablets, minitablets, caplets, pellets, or particles.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, reaction products, and the like. Furthermore, the terms “first,” “second,” and the like, herein do not denote any order, quantity, or importance, but rather are used to denote one element from another. The terms “a” and “an” and “the” herein do not . . . denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “±10%” means that the indicated measurement can be from an amount that is minus 10% to an amount that is plus 10% of the stated value. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs.

Unless otherwise specified herein, any reference to standards, regulations, testing methods and the like, such as USP, refer to the standard, regulation, guidance or method that is in force at the time of filing of the present application.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

What is claimed is:

1. A composition for oral administration comprising a selected phosphodiesterase-5 (PDE5) inhibitor and an additional active agent that reduces or eliminates a side effect associated with the selected PDE5 inhibitor, wherein release of the additional active agent is controlled so that the additional active agent can act at a time and for a time period when the selected PDE5 inhibitor related side effect is expected to occur in the absence of the additional active agent.

2. The composition of claim 1 wherein release of the additional active agent is correlated with half-life of the selected PDE5 inhibitor.

3. The composition of claim 1 wherein the additional active agent is an analgesic, an antacid, an anti-migraine agent, a H2 blocker, a proton pump inhibitor, an anti-gas agent, or a combination thereof.

4. The composition of claim 1, wherein the PDE5 inhibitor is avanafil, sildenafil, tadalafil, vardenafil, lodenafil, udenafil, mirodenafil, a pharmaceutically acceptable salt thereof, or a combination thereof.

5. The composition of claim 1, wherein the PDE5 inhibitor is formulated for immediate release and the additional active agent is formulated for immediate release or controlled release.

6. The composition of claim 5, wherein the controlled release is achieved with a controlled release matrix, a functional coating, or a combination thereof; and wherein the controlled release is extended release, delayed release, pulsed release, or a combination thereof.

7. The composition of claim 1, wherein the PDE5 inhibitor is formulated for controlled release and the additional active agent is formulated for immediate release or controlled release.

8. The composition of claim 7, wherein the PDE5 inhibitor is formulated for immediate release and the additional active agent is formulated for immediate release or controlled release.

9. The composition of claim 1 formulated as a solid, semisolid, or liquid formulation.

10. The composition of claim 1 formulated as a tablet, a capsule, a pellet, a film, a sachet, a powder, a chewable gummy, a solution, a suspension, or an emulsion.

11. A method for treating a condition treatable with a PDE5 inhibitor therapy, said method comprising orally administering to a subject in need thereof the composition of claim 1.

12. The method of claim 11 wherein release of the additional active agent of the composition is correlated with half-life of the selected PDE5 inhibitor of the composition.

13. The method of claim 11 wherein the additional active agent of the composition is an analgesic, an antacid, an anti-migraine agent, a H2 blocker, a proton pump inhibitor, an anti-gas agent, or a combination thereof.

14. The method of claim 11 wherein the PDE5 inhibitor of the composition is avanafil, sildenafil, tadalafil, vardenafil, lodenafil, udenafil, mirodenafil, a pharmaceutically acceptable salt thereof, or a combination thereof.

15. The method of claim 11 wherein the condition being treated is erectile dysfunction, benign prostatic hyperplasia, idiopathic pulmonary hypertension/pulmonary arterial hypertension (PAH), premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof.

16. A method for increasing patient compliance with treatment with a selected PDE5 inhibitor, said method comprising orally administering to a patient the composition of claim 1.

17. The method of claim 16 wherein release of the additional active agent of the composition is correlated with half-life of the selected PDE5 inhibitor of the composition.

18. The method of claim 16 wherein the additional active agent of the composition is an analgesic, an antacid, an anti-migraine agent, a H2 blocker, a proton pump inhibitor, an anti-gas agent, or a combination thereof.

19. The method of claim 16 wherein the PDE5 inhibitor of the composition is avanafil, sildenafil, tadalafil, vardenafil, lodenafil, udenafil, mirodenafil, a pharmaceutically acceptable salt thereof, or a combination thereof.

20. The method of claim 16 wherein the patient is being treated for erectile dysfunction, benign prostatic hyperplasia, idiopathic pulmonary hypertension/pulmonary arterial hypertension (PAH), premature ejaculation associated with erectile dysfunction, high altitude illness, penile rehabilitation after radical prostatectomy, angina pectoris, heart failure, stroke, peripheral neuropathy, male infertility, peripheral arterial disease, diabetic nephropathy, or a combination thereof.
